(19) United States
(12) Patent Application Publication
(10) Pub. No.: US 2015/0211199 A1
(43) Pub. Date: Jul. 30, 2015

(54) DEVICE AND PROCESS TO MEASURE GROUND STIFFNESS FROM COMPACTORS

(71) Applicant: Caterpillar Inc., Peoria, IL (US)

(72) Inventors: PAUL CORCORAN, WASHINGTON, IL (US); LIQUN CHI, PEORIA, IL (US)

(73) Assignee: Caterpillar Inc., Peoria, IL (US)

(21) Appl. No.: 14/163,900

(22) Filed: Jan. 24, 2014

(51) Int. Cl. E02D 1/00; G01N 33/24; E02D 3/026; G01N 3/32; E01C 19/28; E01C 19/26

(52) U.S. Cl. CPC E02D 1/00; E01C 19/288; E01C 19/26; E02D 3/026; G01N 3/32; G01N 33/24

(57) ABSTRACT

This disclosure describes a system and method to calculate a ground stiffness value. The system includes at least one sensor configured to sense at least one of a vertical acceleration, vertical displacement, and a force of a compactor component and a processor configured to calculate a force at a drum-ground interface in response to the at least one sensor and further configured to calculate a drum displacement in response to the at least one sensor. The processor is further configured to determine ground stiffness using the calculated force at the drum-ground interface and the drum displacement.

### TECHNICAL FIELD

The disclosure relates generally to a method for measuring the stiffness of a material while operating a compactor. More specifically, the disclosure relates to measuring the ground stiffness while operating a compactor machine based on contact force and ground displacement.

### BACKGROUND

A wide variety of different compacting systems are used in the preparation of earthen fill and compaction of other materials in civil engineering projects such as construction, road building, and landfill activities. Self-propelled two-wheel and four-wheel compactors, tow-behind systems, and others are well known and widely used. Engineers have recognized for many years that the capacity of substrate materials to remain stable over time, support loads or serve as a barrier to liquids, as well as other properties, can depend in significant part upon compacting a given material to a certain compaction state. Simply passing a compactor over a work area will tend to increase the relative compaction state, and thus the stiffness, of the resident material. Thus, to some extent compactor coverage is one metric which has been used to enable an operator or site manager to estimate that a target ground stiffness has been achieved. While knowing how many times a compactor has traversed a given region of a work area can be useful information, many modern compaction projects require amore sophisticated understanding of the actual compaction state or stiffness of a material.

Different materials may have widely varying “compaction responses” or changes in properties resulting from coverage with a compactor machine. For instance, sandy or granular soils tend to exhibit a different change in relative stiffness than do soils high in clay content each time a compactor is passed over a given region. Local variations in material composition or moisture content within a work area, as well as changes in moisture content over time can also result non-uniformity stiffness even where compactor coverage has been uniform. In addition to project success depending upon satisfaction of stiffness specifications, payments or bonuses to contractors can also be based on the quality and timeliness of a particular compaction job. Like many heavy-duty construction machines, compactors can be quite expensive to operate, and thus unnecessary work or remedial actions create undesired expense. For the foregoing and other reasons, there is often a premium on machines and/or operators capable of doing enough work with a compactor system to meet a predefined goal, but avoiding any substantial wasted effort.

To this end, various strategies are known which provide information to an operator or site manager which is indicative of the stiffness of a material apart from merely how many times the material has been traversed by a compactor. Intelligent compaction technology is growing in acceptance and offered by multiple manufacturers for determining ground stiffness. Intelligent compaction measures generally fall into two categories: those based on frequency response and those based on a calculated force response. Measures based on frequency response are presented as an index whereas measures based on a calculated force response are presented as a ground stiffness value. While calculated force response measures are preferred over frequency response measures, calculated force response measures are subject to significant uncertainty due to impacts on the measured value and calculation of variables other than just ground stiffness. U.S. Pat. No. 8,057,124 B2, assigned to Wacker Neuson Produktion GmbH & Co., discloses a method and device for measuring soil parameters. It uses an approximation of the actual gradient of the contact force and a contact surface parameter that takes into account the geometry and shape of the contact surface to calculate a dynamic modulus of deformation. However, this modulus is a dimensionless value. Because it is a dimensionless value, rather than an industry standard engineering value, its utility is limited.

Thus, a device and method to reduce the degree of uncertainty is needed to ensure compaction to a desired stiffness.

### SUMMARY

In one aspect, a system for calculating ground stiffness using a vibratory compactor includes at least one sensor configured to sense at least one of a vertical acceleration, vertical displacement, and a force of a compactor component, a processor configured to calculate a force at a drum-ground interface in response to the at least one sensor and further configured to calculate a drum displacement in response to the at least one sensor, and the processor further configured to determine ground stiffness using the calculated force at the drum-ground interface and the drum displacement.

In another aspect, a method for calculating ground stiffness using a vibratory compactor includes sensing and calculating a drum-ground contact force with at least one sensor, sensing and calculating a vertical drum displacement from the at least one sensor, and determining ground stiffness based on the drum-ground contact force and the vertical drum displacement with a processor based on the drum-ground contact force and the vertical drum displacement.

In yet another aspect, a system for calculating ground stiffness using a vibratory compactor includes means for sensing and calculating a drum-ground contact force with at least one sensor, means for sensing and calculating a vertical drum displacement from the at least one sensor, and means for determining ground stiffness based on the drum-ground contact force and the vertical drum displacement.

### BRIEF DESCRIPTION OF THE DRAWING(S)

FIG. 1 is a side view of a compactor system, according to one aspect of the disclosure.

FIG. 2 is a pictorial view of a display illustrating a mapped compaction state.

FIG. 3 is a graph depicting the contact force between the drum and the ground during downward compaction and upward retraction according to one aspect of the disclosure.

FIG. 4 is a block diagram of the components needed to carry out one aspect of the disclosure.

FIG. 5 is a free body diagram depicting the various forces and movements while compacting in one aspect of the disclosure.

FIG. 6 is a flowchart depicting a method of calculating the substrate compaction and determining whether the substrate has been sufficiently compacted, according to one aspect of the disclosure.

FIG. 7 is a graph illustrating the contact force between the drum and the ground during downward compaction and upward retraction when the vibratory compactor is operating in a double jump mode.

### DETAILED DESCRIPTION

The disclosure relates generally to a system and method for determining a ground stiffness measure while the ground is being compacted with a compactor. Loose material, characterized as material which can be further packed or densified, is disposed over the surface. As the compactor machine travels over the surface, forces generated by the compactor machine and imparted to the surface, acting in cooperation with the weight of the machine, compress the loose material to a state of greater stiffness and density. The compactor machine may make one or more passes over the surface to provide a desired level of ground stiffness. In one intended application, the loose material may be freshly deposited asphalt that is to be compacted into roadways or similar hardtop surfaces. In other applications, the material may be soil, gravel, sand, land fill trash, concrete or the like.

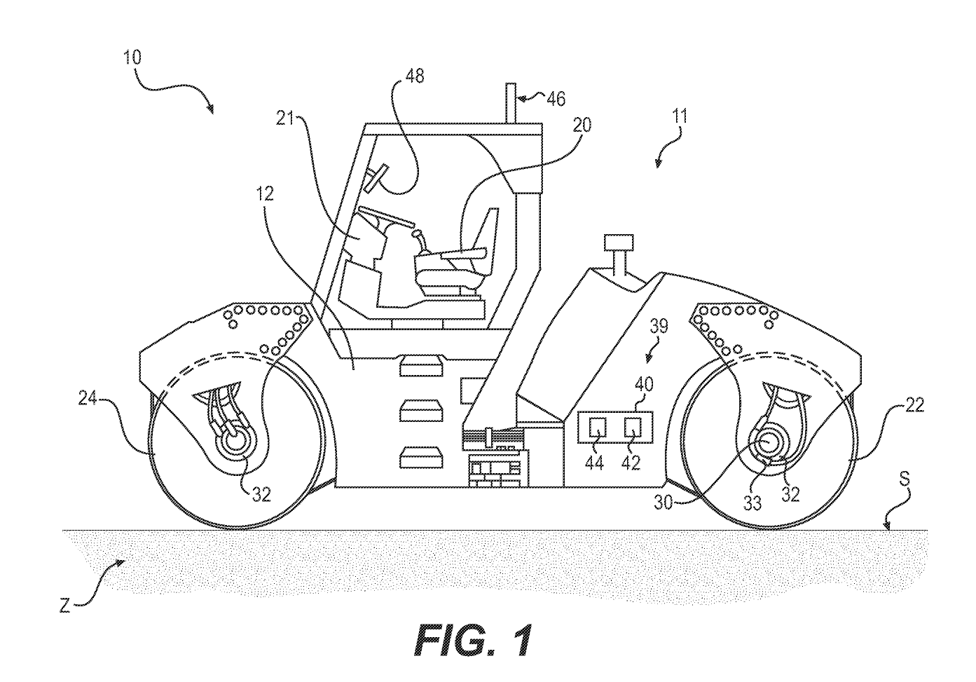

FIG. 1 is a side view of a compactor system, according to one aspect of the disclosure. In particular, FIG. 1 illustrates an exemplary compactor system 10 of the self-propelled type that can travel over a surface S under its own power that may implement aspects of the disclosure. Other types of compactors are contemplated to implement the disclosed process and device including self-propelled two-wheel and four-wheel compactors and tow-behind systems. The compactor system 10 includes a compactor machine 11 that includes a body or frame 12 that inter-operatively connects and associates the various physical and structural features that enable the compactor machine 11 to function. These features may include an operator cab 20 that is mounted on top of the frame 12 from which an operator may control and direct operation of the compactor machine 11. Additionally, a steering feature 21 and similar controls may be located within the operator cab 20. To propel the compactor machine 11 over the surface S, a power system (not shown), such as an internal combustion engine, can also be mounted to the frame 12 and can generate power that is converted to physically move the compactor machine 11. One or more other implements 36 may be connected to the machine. Such implements 36 may be utilized for a variety of tasks, including, for example, loading, lifting, and brushing, and may include, for example, buckets, forked lifting devices, brushes, grapples, cutters, shears, blades, breakers/hammers, augers, and others.

To enable physical motion of the compactor machine 11, the illustrated compactor machine 11 includes a first roller drum 24 and a second roller drum 22 that are in rolling contact with the surface S. For reference purposes, the compactor machine 11 can have a typical direction of travel such that the first roller drum 24 may be considered the forward roller drum and the second roller drum 22 considered the rearward roller drum. The first (forward) and second (rearward) roller drums 24, 22 can be cylindrical structures that are rotatably coupled to and can rotate with respect to the frame 12. Because of their forward and rearward positions and their dimensions, the first (forward) and second (rearward) roller drums 24, 22 support the frame 12 of the compactor machine 11 above the surface S and allow it to travel over the surface S. The first (forward) and second (rearward) roller drums 24, 22 are oriented generally transverse or perpendicular to the direction of travel of the compactor machine 11. It should be appreciated that because the compactor machine 11 is steerable, the forward direction of travel may change bearing during the course of operation but can be typically assessed by reference to the direction of movement of the first (forward) roller drum 24. In the illustrated aspect, to transfer motive power from the power system to the surface S, the power system can operatively engage and rotate the second (rearward) roller drum 22 through an appropriate power train.

To facilitate control and coordination of the compactor machine 11, the compactor machine 11 may include a controller 39 such as an electronic control unit 40. The main unit of the controller 39 may be located in the operator cab 20 for access by the operator and may communicate with the steering feature 21, the power system, and with various other sensors and controls on the compactor machine 11. While the controller 39 illustrated in FIG. 1 is represented as a single unit, in other aspects the controller 39 may be distributed as a plurality of distinct but interoperating units, incorporated into another component, or located at a different location on or off the compactor machine 11.

The controller 39 may include a sensor 32 configured to sense a parameter indicative of the acceleration, velocity, and/or force of a component of the compactor machine 11. The components may include the first (forward) and/or second (rearward) roller drums 24, 22, the compactor frame 12, or the like. Sensor 32 may include a signal transducer configured to sense a transmitted signal, or component of a transmitted signal. For example, the signal reflected by surface S. In the illustrated aspect, a single sensor 32 is shown coupled with and resident on first (forward) roller drum 24. In other aspects, additional sensors such as a rear sensor (not shown) associated with second (rear) roller drum 22, individual sensors located in proximity to the first (forward) and/or second (rearward) roller drums 24, 22, or separate sensors for measuring acceleration of the first (forward) and second (rearward) roller drums 24, 22 and the compactor frame 12 may be used.

For example, one sensor 32 may sense the vertical acceleration of the first (forward) and/or second (rearward) roller drums 24, 22 and a second sensor may detect the vertical acceleration of the compactor frame 12. These sensors 32 may be located proximate to each other but they need not be. For example, a sensor 32 sensing the vertical acceleration of a drum 22, 24 may be located on the first (forward) roller drum 24 while a second sensor 32 sensing the vertical acceleration of the compactor frame 12 may be located on the second (rearward) roller drum 22. Additionally, there may be more than one of each type of sensor 32 located on the compactor machine 11. For example, there may be a sensor 32 sensing the vertical acceleration of the first (forward) roller drum 24 and a second sensor sensing the vertical acceleration of the second (rearward) roller drum 22. While the acceleration of both the drum and the compactor frame may be used, the drum acceleration only may be used as the primary signal. The sensor 32 can be an accelerometer. The accelerometer used can be any type of accelerometer. Such accelerometers include, but are not limited to, laser accelerometers, low frequency accelerometers, bulk micromachined capacitive accelerometers, strain gauge accelerometers, and bulk micromachined piezoelectric accelerometers among others.

In one aspect, the sensor 32 may sense force. In this case, the sensor 32 may be, but is not limited to, a load cell, a strain gauge, or the like.

In one aspect, the sensor 32 may be located at or close to a center point of axle 30, at or close to a longitudinal centerline of frame 12. The transmitted signal may include a sonic signal, an RF signal, or a laser signal, for example, transmitted via a transmitter 34 mounted with sensor 32 in a housing 38. Sensor 32 may include a non-contact sensor such as the examples noted above.

Controller 39 may further include a phase sensor 33. Phase sensor 33 measures the phase angle of a vibratory force imparted by the first (forward) and/or second (rearward) roller drums 24, 22 to the ground. The phase may be measured in real time.

The mechanism generating the vibratory force may be a mechanical vibratory system, which rotates one or more eccentric masses to generate the vibratory force, or the vibratory force may be generated by an electrically-driven vibratory system. The phase angle needs to be measured in real-time.

Controller 39 may further include a location sensor 46 resident on compactor machine 11 which receives global or local positioning data used in establishing and tracking geographic position of compactor machine 11 within a work area. In one aspect, further described herein, data received via the location sensor 46 may be linked with data received from sensor 32 to map position data of the compactor system 10 received via a location sensor 46 to ground stiffness, for purposes which will be apparent from the following description.

Controller 39 may further include an electronic control unit 40 which includes at least one data processor 42 and a computer readable memory 44. Electronic control unit 40 may be coupled with sensor 32, and also with location sensor 46, and may be configured to output a signal responsive to inputs from sensor 32, as further described herein. A display 48 also may be coupled with electronic control unit 40 and may be positioned in the operator cab 20 to display various data to an operator relating to machine position, ground stiffness, or still other parameters. In the illustrated aspect, the controller 39 is resident on compactor machine 11. It should be appreciated that in other aspects, controller 39 or parts thereof might be located remotely from the compactor machine 11, such as at an on-site or offsite management office. In such an aspect, data gathered relating to position of compactor machine 11 and ground stiffness data might be transmitted to a remote computer, processed, and control commands sent to the compactor machine 11 to direct an operator to take or forego certain actions, or to direct compactor machine 11 to autonomously take or forego certain actions. Taking actions in response to the ground stiffness data and position data might include commencing travel of compactor machine 11 within a work area, stopping travel of compactor machine 11 within a work area, or redirecting or otherwise changing a planned compactor travel path or coverage pattern. Computer readable memory 44 may store computer executable code including a control algorithm for determining an absolute ground stiffness value of material substrate Z and/or determining a change in ground stiffness, responsive to inputs from sensor 32, and the same or another algorithm for controlling or directing operation of compactor machine 11.

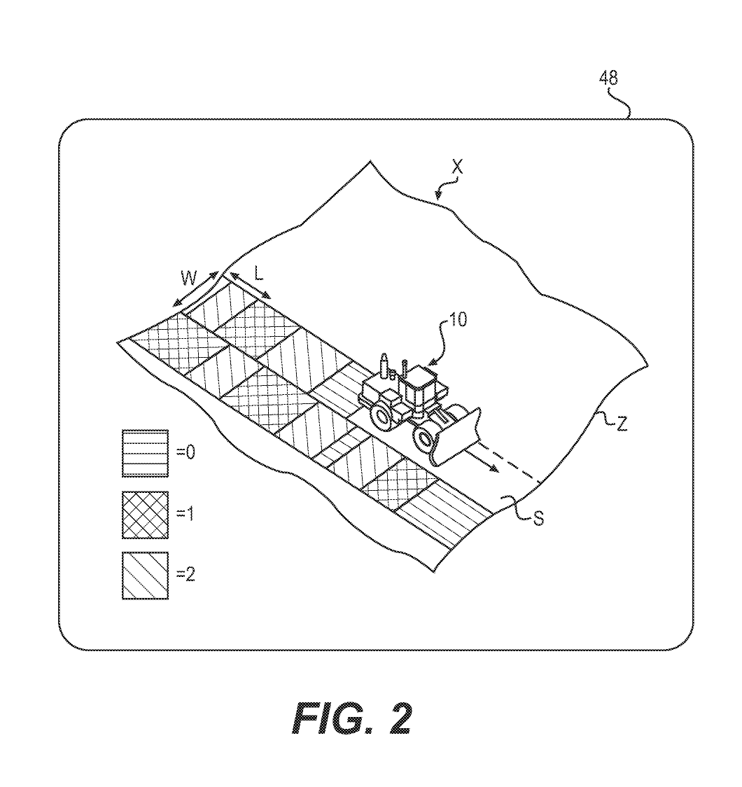

FIG. 2 is a pictorial view of a display illustrating a mapped compaction state. The work area has been fragmented into smaller work areas of length L and width W. The smaller work areas have been filled in by different patterns, depending upon the substrate compaction state. The fragmented work areas that have been filled in with horizontal lines indicate that that area has yet to be compacted. The areas that have been filled in with crisscross lines indicate that the compactor has compacted the area but that the substrate has not reached the desired compaction level. And, work areas with diagonal lines indicate that the area has been compacted to a sufficient compaction level as determined by the disclosed process and device. Such a map can be generated using the location sensor 46 on the compactor machine 11 and the method and device disclosed herein for calculating the ground stiffness.

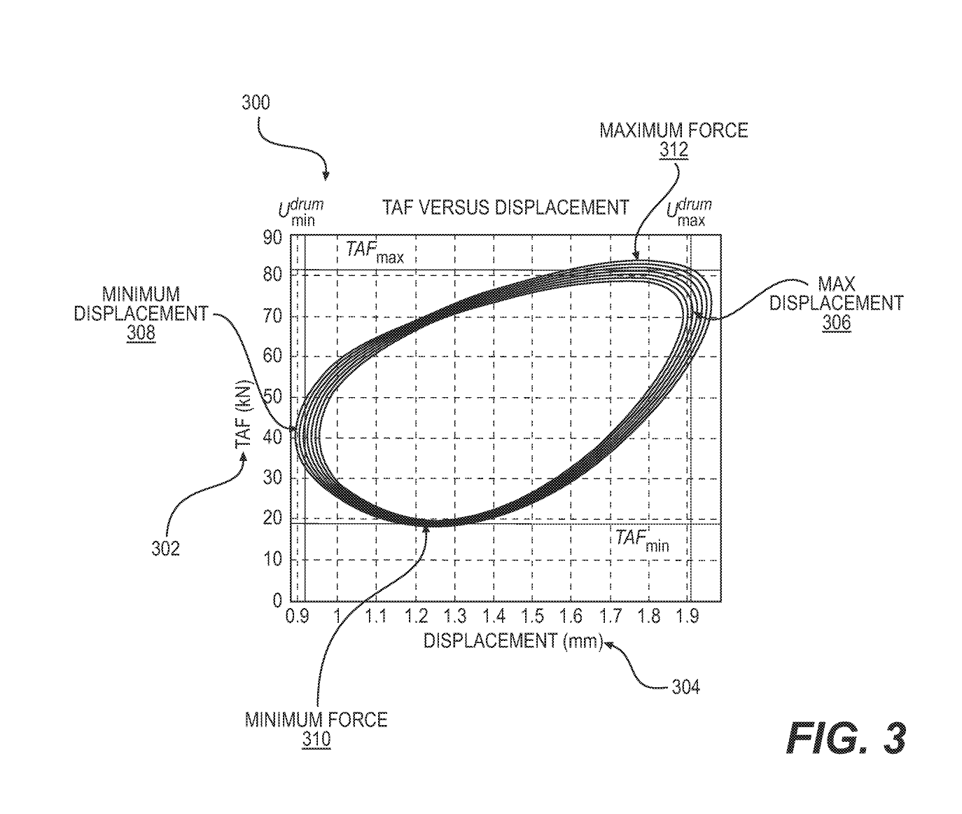

FIG. 3 is a graph 300 depicting the contact force between the first (forward) and/or second (rearward) roller drums 24, 22 and the ground during downward compaction and upward retraction according to one aspect of the disclosure. In particular, FIG. 3 is a graph showing the total applied force (TAF) that the first (forward) and/or second (rearward) roller drums 24, 22 and the compactor frame 12 apply to the ground compared to the displacement of the first (forward) and/or second (rearward) roller drums 24, 22. In particular, this graph 300 shows multiple cycles of drum operation 302, 304. The TAF starting at roughly 20 kN at a displacement of roughly 1.2 mm is the point of minimum force 310 and tracing the graph to the top to about 82 kN at a displacement of about 1.8 mm, the point of maximum force 312, represents the loading portion of the compaction process. Starting at the point of maximum displacement 306, and continuing to the left to the point of minimum force 310 represents the unloading portion of the compaction process. It should be noted that the values in FIG. 3 and described hereinabove are merely exemplary.

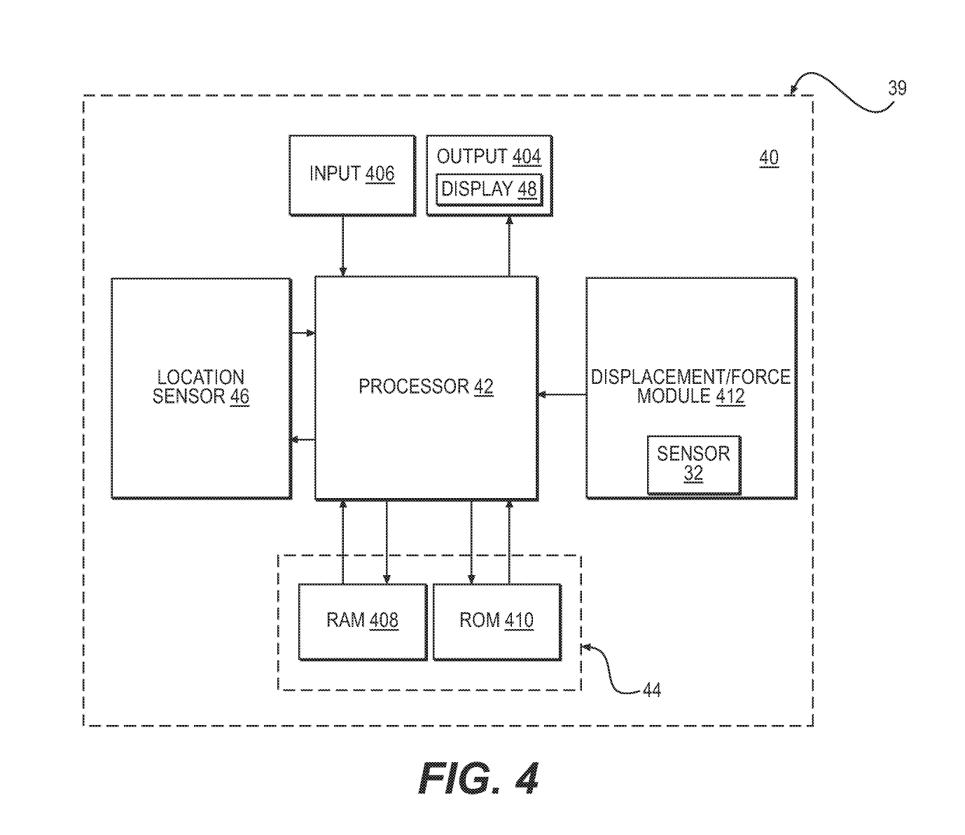

FIG. 4 is a block diagram of the components needed to carry out one aspect of the disclosure. The controller 39 may include an input device 406, an output 404, a processor 42, a random access memory (RAM) 408, a read-only memory (ROM) 410, a displacement/force module 412, and the location sensor 46. A user can use an input device 406 to enter in a minimum ground stiffness value that must be achieved for the particular work area. The input device 406 can be located on the compactor machine 11 or it may be located remotely. For example, the input device 406 may be a keyboard located in the operator cab 20. Alternatively, the input device 406 could be a mobile device, such as a smartphone, a tablet, a personal digital assistant (PDA), wearable devices such as a smartwatch, or a laptop computer. Non-mobile devices, such as desktop computers and computer servers, may also be used and may communicate over a communication channel to the controller 39.

The displacement/force module 412 collects information from the sensor 32 indicative of the vertical acceleration, velocity, and/or force of the first (forward) and/or second (rearward) roller drums 24, 22 and/or the vertical acceleration, velocity, and/or force of the compactor frame 12. One or more sensors 32 may accomplish these functions as described above. The displacement/force module 412 may include an A/D converter, driver, and the like to capture the output from sensor 32.

Random access memory (RAM) 408 may store various digital files including the values sensed by the sensor 32. The RAM 408 can be any suitable computer-readable medium. Examples of RAM 408 include, but are not limited to, dynamic random access memory (DRAM), static random access memory (SRAM), ferroelectric random access memory (FRAM), resistive random access memory (RRAM), and diode memory among others. The RAM 408 may provide the sensed values to the processor 42 so that the processor 42 can calculate the ground stiffness values based on the values. The RAM 408 can also store calculated ground stiffness values.

The read-only memory (ROM) 410 in FIG. 4 may store various digital files. Examples of ROM 410 include, but are not limited to, programmable read-only memory (PROM), erasable programmable read-only memory (EPROM), electrically erasable programmable read-only memory (EEPROM), which includes electrically alterable read-only memory (EAROM) and flash memory, and optical storage, such as CD-ROMs. The RAM 408 and/or ROM 410 may store the algorithm the processor 42 uses to calculate the ground stiffness value.

Processor 42 utilizes the values sensed by the sensor 32 that may be stored in the RAM 408 to calculate a ground stiffness value using the algorithm stored in the ROM 410 or RAM 408. The processor 42 may compare the calculated ground stiffness value to the minimum ground stiffness value the user input. If the calculated ground stiffness value meets or exceeds the minimum ground stiffness value, the processor 42 may send a signal to the output 404 communicating that the ground has been sufficiently compacted. However, if the calculated ground stiffness value does not meet or exceed the minimum ground stiffness value, then the processor 42 may send a signal to the output 404 communicating that further compaction is required. Examples of processors include computing devices and/or dedicated hardware as defined herein, but are not limited to, one or more central processing units and microprocessors.

The output 404 may be configured to communicate information to the user including whether the ground has reached the minimum ground stiffness required for the work area. The output 404 can include a display 48. Such displays include, but are not limited to, cathode ray tubes (CRT), light-emitting diode display (LED), liquid crystal display (LCD), organic light-emitting diode display (OLED) or a plasma display panel (PDP). Such displays can also be a touchscreen and may incorporate aspects of the input device 406. The output 404 may also include a transceiver. The transceiver communicates over a communication channel as defined herein.

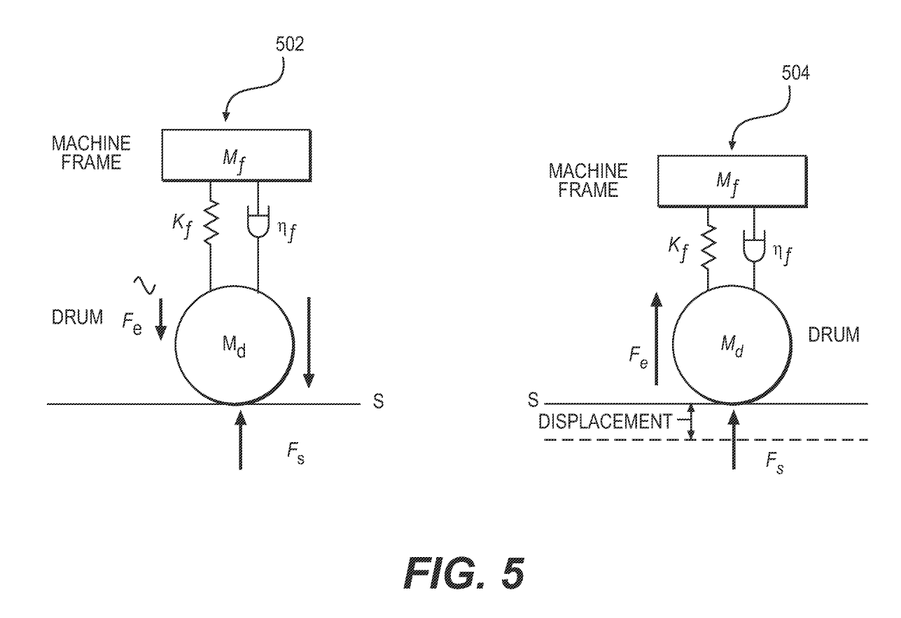

FIG. 5 is a free body diagram depicting the various forces and movements while compacting in one aspect of the disclosure. In particular, FIG. 5 shows a free body diagram illustrating the mechanical components and the Newtonian forces present while the compactor system 10 compacts the surface of a work area. A portion of the compactor frame 12 mass M_{f} and the mass of a drum, either the first (forward) roller drum 24 or the second (rearward) roller drum 22, M_{d}, together with their respective accelerations, combine to form a downward force, Fₑ, on the surface S in order to further compact the surface S. The ground reaction force is represented by Fₛ. The spring-mass-damper system, K_{f} and η_{f}, represent the damping mechanism properties of the compactor machine 11. The left-hand illustration 502 in FIG. 5 represents the compactor system 10 when it is loading, or compressing, the ground. This is illustrated by the downward pointing force Fₑ. The right-hand illustration 504 in FIG. 5 represents the compactor system 10 when it is unloading from the ground. This is illustrated by the upward pointing force Fₑ. The displacement is the difference in the first (forward) and/or second (rearward) roller drum 24, 22 height when the first (forward) and/or second (rearward) roller drum 24, 22 is compressing the ground and the first (forward) and/or second (rearward) roller drum 24, 22 height when the first (forward) and/or second (rearward) roller drum 24, 22 is not compressing the ground.

### INDUSTRIAL APPLICABILITY

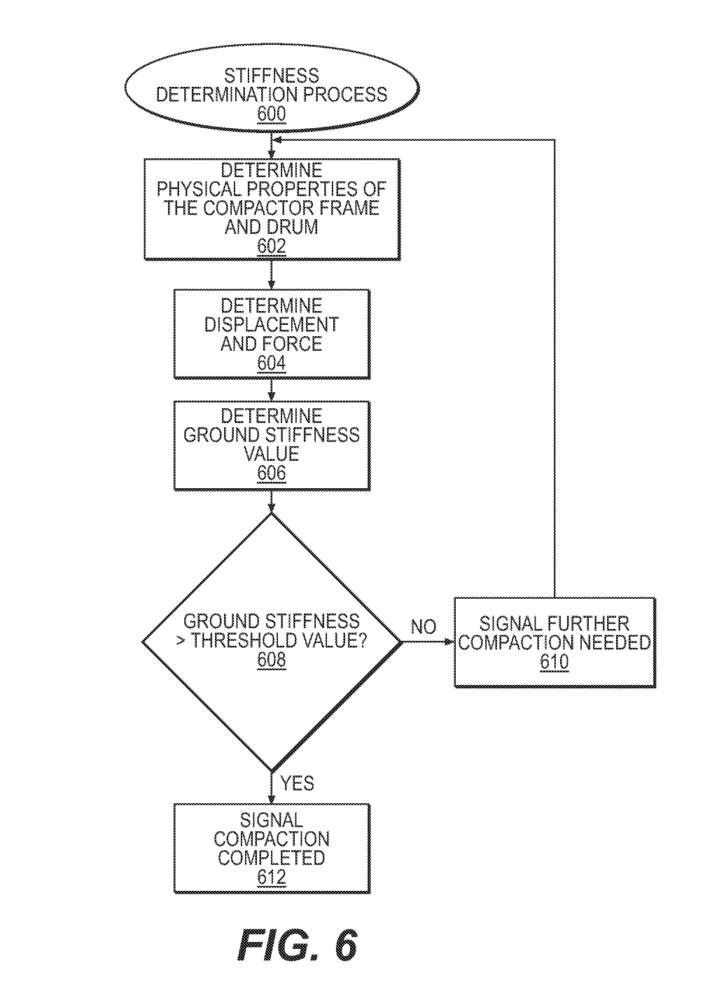

FIG. 6 is a flowchart depicting a method of calculating the substrate compaction and determining whether the substrate has been sufficiently compacted, according to one aspect of the disclosure. FIG. 6 illustrates a method of using the disclosed system in order to calculate the ground stiffness value of the ground that is compacted using the compactor machine 11. The equation that may be used to calculate the contact force at the drum-ground interface according to the disclosure is as follows:

Fₛ=(M_{d}+M_{f})g−M_{d}\ddot{z}_{d}−M_{f}\ddot{z}_{f}+Fₑ sin(ω₀t+φ₀) Equation 1

where Fₛ is the contact force, M_{d} is the unsprung mass of the first (forward) and/or second (rearward) roller drum 24, 22, M_{f} is the equivalent frame mass on the drum axle, \ddot{z}_{d} is the vertical acceleration of the first (forward) and/or second (rearward) roller drum 24, 22, \ddot{z}_{f} is the vertical acceleration of the frame, Fₑ is the amplitude of the vibratory force, ω₀ is the angular frequency of the vibratory force, and φ₀ is the phase angle of the vibratory force. As the above equation makes clear, information is needed about the physical properties of the compactor machine 11. Thus, in 602, the system will determine information about the physical properties of the compactor machine 11, such as the mass of the first (forward) and/or second (rearward) roller drums 24, 22, the mass of the compactor frame 12 exerted on the first (forward) and/or second (rearward) roller drums 24, 22, and the like. Other approaches to determining Fₛ are contemplated as well, including direct sensing with the sensor 32 implemented as a force sensor.

In 604, the contact force at the drum-ground interface is calculated using the physical properties gathered in 602, the values sensed by the sensor 32, such as the vertical acceleration of the first (forward) and/or second (rearward) roller drums 24, 22 and the vertical acceleration of the compactor frame 12, and the vibrational properties, if any, of the first (forward) and/or second (rearward) roller drums 24, 22. As noted previously, the vertical acceleration of the first (forward) and/or second (rearward) roller drums 24, 22, rather than the vertical acceleration of the compactor frame 12, provide the primary signal used in the calculation. These values are then used according to Equation 1 to calculate the contact force at the drum-ground interface.

The drum displacement can be calculated by using the drum acceleration values that the sensor 32 has sensed. To calculate a displacement value from an acceleration value, the acceleration value may be integrated twice. In other words, the displacement value can be calculated according to the following formula:

z=z₀+∫₀^{t}[ż₀+∫₀^{t}\ddot{z}₀]dt Equation 2

where z is the displacement of the drums 22, 24, z₀ is the initial vertical displacement of the first (forward) and/or second (rearward) roller drums 24, 22 at the beginning of each sample cycle, ż₀ is the initial vertical velocity of the first (forward) and/or second (rearward) roller drums 24, 22 at the beginning of each sampling cycle, and \ddot{z}₀ is the vertical acceleration of the first (forward) and/or second (rearward) roller drums 24, 22. The initial displacement z₀ does not affect the energy and stiffness calculation so, as a simplification, it can be set to zero. However, the initial vertical velocity ż₀ will affect the energy and stiffness calculations. An initial vertical drum velocity ż₀ is determined by making zero average velocity during a complete sampling cycle, which is normally two or more vibratory cycles. When compacting level ground, the average vertical velocity of the first (forward) and/or second (rearward) roller drums 24, 22 is almost zero. However, when compacting on a slope, the average vertical velocity is not zero. Setting the average vertical velocity to zero will remove the effect created by the slope. Other approaches to determine z are contemplated, including a sensor 32 implemented as a distance sensor.

Once the contact force at the drum-ground interface and the displacement of the first (forward) and/or second (rearward) roller drum 24, 22 is calculated, the ground stiffness value can be calculated at 606. To calculate the ground stiffness value, the information needed is the maximum and the minimum TAF and the maximum and the minimum drum displacement values. The ground stiffness value can be calculated according to the following equation:

$${k}_{dyn} = mean [ \frac{( {TAF}_{max} - {TAF}_{min} )}{{u}_{max}^{drum} - {u}_{min}^{drum}} ] \qquad Equation 3$$

This is a simpler and more robust equation as compared to the prior art. Additionally, the unit of the calculation is force per length, which aligns with the stiffness measure.

Once the ground stiffness value has been calculated, it may be compared to the threshold ground stiffness required for the work area that may be input by the user as indicated in 608. If the calculated ground stiffness value is below the threshold ground stiffness required for the work area, then the processor 42 may send a signal to the output 404 and may display that further compaction is required. The operator can then compact the ground further until the calculated ground stiffness value reaches the threshold ground stiffness value. If, however, the calculated ground stiffness value meets the threshold ground stiffness required for the work area, then the processor 42 may send a signal to the output 404 and may display that no further compaction is needed and the process ends at 612.

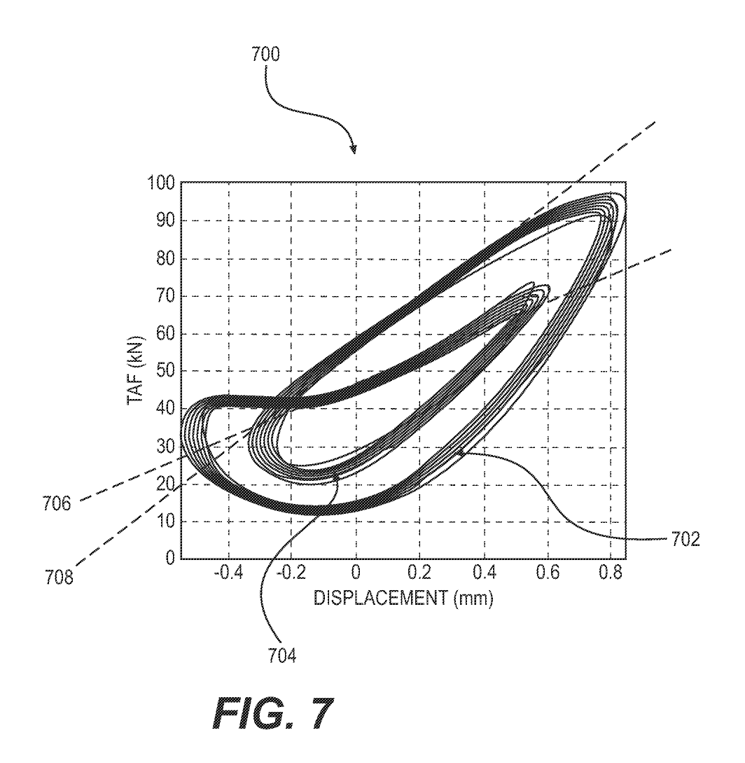

FIG. 7 is a graph illustrating the contact force between the first (forward) and/or second (rearward) roller drums 24, 22 and the ground during downward compaction and upward retraction when the compactor is operating in a double jump mode. FIG. 7 shows the effect of double jumping on the TAF versus drum displacement graph 700. The larger, outer shape 702 is the true TAF versus drum displacement while the smaller, inner shape 704 is the TAF versus drum displacement when double jumping is present. In some prior art approaches, only a loading slope was used to calculate the ground stiffness. However, as illustrated in FIG. 7, when double jumping is present, there are actually two loading slopes 706, 708. Thus, when double jumping is present, the prior art methods calculate the ground stiffness value using both slopes 706, 708: the true loading slope 708 and the false loading slope 706 caused by the double jumping. The result, then, is an inaccurate measurement of ground stiffness. However, the disclosure overcomes this limitation by not relying on the loading slope. Instead, the disclosure utilizes the maximum and the minimum drum-ground contact force and the maximum and the minimum drum displacement. Thus, the distortion caused by double jumping is removed and a more accurate ground stiffness value is achieved. The disclosed method and device offer a more accurate calculation of the ground stiffness value, especially in situations where the compactor machine 11 is operating in a double jump mode.

The device and process may include communication channels that may be any type of wired or wireless electronic communications network, such as, e.g., a wired/wireless local area network (LAN), a wired/wireless personal area network (PAN), a wired/wireless home area network (HAN), a wired/wireless wide area network (WAN), a campus network, a metropolitan network, an enterprise private network, a virtual private network (VPN), an internetwork, a backbone network (BBN), a global area network (GAN), the Internet, an intranet, an extranet, an overlay network, a cellular telephone network, a Personal Communications Service (PCS), using known protocols such as the Global System for Mobile Communications (GSM), CDMA (Code-Division Multiple Access), W-CDMA (Wideband Code-Division Multiple Access), Wireless Fidelity (Wi-Fi), Bluetooth, Long Term Evolution (LTE), EVolution-Data Optimized (EVDO) and/or the like, and/or a combination of two or more thereof.

The device and process may be implemented in any type of computing devices, such as, e.g., a desktop computer, personal computer, a laptop/mobile computer, a personal data assistant (PDA), a mobile phone, a tablet computer, cloud computing device, and the like, with wired/wireless communications capabilities via the communication channels.

Further in accordance with various aspects of the disclosure, the methods described herein are intended for operation with dedicated hardware implementations including, but not limited to, PCs, PDAs, semiconductors, application specific integrated circuits (ASIC), programmable logic arrays, cloud computing devices, and other hardware devices constructed to implement the methods described herein.

It should also be noted that the software implementations of the disclosure as described herein are optionally stored on a tangible storage medium, such as: a magnetic medium such as a disk or tape; a magneto-optical or optical medium such as a disk; or a solid state medium such as a memory card or other package that houses one or more read-only (non-volatile) memories, random access memories, or other re-writable (volatile) memories. A digital file attachment to email or other self-contained information archive or set of archives is considered a distribution medium equivalent to a tangible storage medium. Accordingly, the disclosure is considered to include a tangible storage medium or distribution medium, as listed herein and including art-recognized equivalents and successor media, in which the software implementations herein are stored.

The many features and advantages of the disclosure are apparent from the detailed specification, and, thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

We claim:

1. A system for calculating ground stiffness associated with compactor operation, comprising:
at least one sensor configured to sense at least one of a vertical acceleration, vertical displacement, and a force of a compactor component;
a processor configured to calculate a force at a drum-ground interface in response to the at least one sensor and further configured to calculate a drum displacement in response to the at least one sensor; and
the processor further configured to determine ground stiffness using the calculated force at the drum-ground interface and the drum displacement.

2. The system for calculating ground stiffness of claim 1 wherein the processor is further configured to calculate a maximum and a minimum vertical displacement of a drum.

3. The system for calculating ground stiffness of claim 1 wherein the processor is further configured to calculate a maximum and a minimum force at the drum-ground interface.

4. The system for calculating ground stiffness of claim 1 wherein the processor is further configured to calculate a maximum and a minimum vertical displacement of a drum and a maximum and a minimum force at the drum-ground interface.

5. The system for calculating ground stiffness of claim 1 wherein the at least one sensor is configured to sense a vertical acceleration of a compactor drum.

6. The system for calculating ground stiffness of claim 1 wherein the at least one sensor is an accelerometer.

7. The system for calculating ground stiffness of claim 1 further comprising a position sensor resident on a compactor configured to receive global or local positioning data of the compactor.

8. The system for calculating ground stiffness of claim 1, wherein the at least one sensor is configured to sense a vertical acceleration of a compactor.

9. A compactor comprising the system for calculating ground stiffness of claim 1 further comprising at least one drum.

10. A method for calculating ground stiffness associated with compactor operation, comprising:
sensing and calculating a drum-ground contact force with at least one sensor;
sensing and calculating a vertical drum displacement from the at least one sensor; and
determining ground stiffness based on the drum-ground contact force and the vertical drum displacement with a processor based on the drum-ground contact force and the vertical drum displacement.

11. The method of calculating ground stiffness of claim 10, wherein calculating a drum-ground contact force further comprises calculating a maximum and a minimum drum-ground contact force.

12. The method of calculating ground stiffness of claim 10, wherein calculating the vertical drum displacement further comprises calculating a maximum and a minimum vertical drum displacement.

13. The method of calculating ground stiffness of claim 10 wherein calculating a drum-ground contact force further comprises calculating a maximum and a minimum drum-ground contact force and calculating the vertical drum displacement further comprises calculating a maximum and a minimum vertical drum displacement.

14. The method of calculating ground stiffness of claim 10 wherein the at least one sensor sensing and calculating the vertical drum displacement is an accelerometer that senses a vertical acceleration of a drum.

15. A system for calculating ground stiffness associated with compactor operation, comprising:
means for sensing and calculating a drum-ground contact force with at least one sensor;
means for sensing and calculating a vertical drum displacement from the at least one sensor; and
means for determining ground stiffness based on the drum-ground contact force and the vertical drum displacement.

16. The system for calculating ground stiffness of claim 15 wherein the means for sensing and calculating a drum-ground contact force further comprises calculating a maximum and a minimum drum-ground contact force.

17. The system for calculating ground stiffness of claim 15 wherein the means for sensing and calculating a vertical drum displacement further comprises calculating a maximum and a minimum vertical drum displacement.

18. The system for calculating ground stiffness of claim 15 wherein the means for sensing and calculating a drum-ground contact force further comprises calculating a maximum and a minimum drum-ground contact force and wherein the means configured to sense and calculate a vertical drum displacement further comprises calculating a maximum and a minimum vertical drum displacement.

19. The system for calculating ground stiffness of claim 15 wherein the at least one sensor is an accelerometer and senses a vertical acceleration of a drum.

20. A compactor comprising the system for calculating ground stiffness of claim 15 further comprising at least one drum.
